(19) United States
(12) Patent Application Publication
(10) Pub. No.: US 2020/0352661 A1
(43) Pub. Date: Nov. 12, 2020

(54) ROBOTIC STAND FOR ROBOTIC SURGICAL SYSTEM

(71) Applicant: Medrobotics Corporation, Raynham, MA (US)

(72) Inventors: Thomas Calef, Bridgewater, MA (US); James Wilson, Norwood, MA (US); Alexander Patt Broerman, Somerville, MA (US); Nathanial Sharpe, Cambridge, MA (US); Neil Bacon, Milton, MA (US); Kyle Benton, Roxbury Crossing, MA (US); Stuart D. Perry, Wellesley Hills, MA (US); Guarav Rohatgi, Waltham, MA (US)

(21) Appl. No.: 16/960,156

(22) PCT Filed: Jan. 3, 2019
(86) PCT No.: PCT/US19/12158

§ 371 Date: Jul. 6, 2020

Related U.S. Application Data
(60) Provisional application No. 62/614,223, filed on Jan. 5, 2018.
(60) Provisional application No. 62/614,224, filed on Jan. 5, 2018.

(51) Int. Cl. A61B 34/30; A61B 90/50; A61B 50/24

(52) U.S. Cl. CPC A61B 34/30; A61B 90/50; A61B 1/018; A61B 2034/301; A61B 50/24

(57) ABSTRACT

A system for performing a medical procedure on a patient includes an articulating probe assembly and at least one tool. The articulating probe assembly comprises an inner probe comprising multiple articulating inner links, an outer probe surrounding the inner probe and comprising multiple articulating outer links, and at least two working channels that exit a distal portion of the probe assembly. The at least one tool is configured to translate through one of the at least two working channels. A robotic stand positions the articulating probe relative to the patient.

### RELATED APPLICATIONS

This application claims the benefit of U.S. Provisional Application No. 62/613,899, filed Jan. 5, 2018, the content of which is incorporated herein by reference in its entirety.

This application claims the benefit of U.S. Provisional Application No. 62/614,223, filed Jan. 5, 2018, the content of which is incorporated herein by reference in its entirety.

This application claims the benefit of U.S. Provisional Application No. 62/614,224, filed Jan. 5, 2018, the content of which is incorporated herein by reference in its entirety.

This application claims the benefit of U.S. Provisional Application No. 62/614,228, filed Jan. 5, 2018, the content of which is incorporated herein by reference in its entirety.

This application claims the benefit of U.S. Provisional Application No. 62/614,225, filed Jan. 5, 2018, the content of which is incorporated herein by reference in its entirety.

This application claims the benefit of U.S. Provisional Application No. 62/614,240, filed Jan. 5, 2018, the content of which is incorporated herein by reference in its entirety.

This application claims the benefit of U.S. Provisional Application No. 62/614,235, filed Jan. 5, 2018, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, PCT Publication No. WO2015/102939, the content of which is incorporated herein by reference in its entirety.

This application is related to U.S. patent application Ser. No. 14/892,750, filed Nov. 20, 2015, U.S. Publication No. 2016/0256226, now U.S. Pat. No. 10,004,568 issued on Jun. 26, 2018, the content of which is incorporated herein by reference in its entirety.

This application is related to U.S. patent application Ser. No. 15/899,826, filed Feb. 20, 2018, U.S. Publication No. 2018/0250095 the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, PCT Publication No. WO2012/054829, the content of which is incorporated herein by reference in its entirety.

This application is related to U.S. patent application Ser. No. 13/880,525, filed Apr. 19, 2013, U.S. Publication No. 2014/0005683, now U.S. Pat. No. 8,992,421, issued on Mar. 31, 2015, the content of which is incorporated herein by reference in its entirety.

This application is related to U.S. patent application Ser. No. 14/587,166, filed Dec. 31, 2014, U.S. Publication No. 2015/0313449, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US2012/040414, filed Jun. 1, 2012, PCT Publication No. WO2012/167043, the content of which is incorporated herein by reference in its entirety.

This application is related to U.S. patent application Ser. No. 14/119,316, filed Nov. 21, 2013, U.S. Publication No. 2014/0094825, the content of which is incorporated herein by reference in its entirety.

This application is related to U.S. Provisional Application No. 62/504,175, filed May 10, 2017, the content of which is incorporated herein by reference in its entirety.

This application is related to PCT Application No. PCT/US2018/031774, filed May 9, 2018, PCT Publication No. WO2018/0020898, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, PCT Publication No. WO2012/078309, the content of which is incorporated herein by reference in its entirety.

This application is related to U.S. patent application Ser. No. 13/884,407, filed May 9, 2013, U.S. Publication No. 2014/0012288, now U.S. Pat. No. 9,649,163, issued on May 16, 2017, the content of which is incorporated herein by reference in its entirety.

This application is related to U.S. patent application Ser. No. 15/587,832, filed May 5, 2017, U.S. Publication No. 2018/0021095, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US2012/032279, filed Apr. 5, 2012, PCT Publication No. WO2012/138834, the content of which is incorporated herein by reference in its entirety.

This application is related to U.S. patent application Ser. No. 14/008,775, filed Sep. 30, 2013, U.S. Publication No. 2014/0046305, now U.S. Pat. No. 9,962,179, issued on May 8, 2018, the content of which is incorporated herein by reference in its entirety.

This application is related to U.S. patent application Ser. No. 14/944,665, filed Nov. 18, 2015, U.S. Publication No.: 2016/0066938, the content of which is incorporated herein by reference in its entirety.

This application is related to U.S. patent application Ser. No. 14/945,685, filed Nov. 19, 2015, U.S. Publication No. 2016/0066939, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US2012/054802, filed Sep. 12, 2012, PCT Publication No. WO2013/039999, the content of which is incorporated herein by reference in its entirety.

This application is related to U.S. patent application Ser. No. 14/343,915, filed Mar. 10, 2014, U.S. Publication No. 2014/0371764, now U.S. Pat. No. 9,757,856, issued on Sep. 12, 2017, the content of which is incorporated herein by reference in its entirety.

This application is related to U.S. patent application Ser. No. 15/064,043, filed Mar. 8, 2016, U.S. Publication No. 2016/0262840, now U.S. Pat. No. 9,572,628, issued on Feb. 21, 2017, the content of which is incorporated herein by reference in its entirety.

This application is related to U.S. patent application Ser. No. 15/684,268, filed Aug. 23, 2017, U.S. Publication No. 2017/0368681, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, PCT Publication No. WO2012/015659, the content of which is incorporated herein by reference in its entirety.

This application is related to U.S. patent application Ser. No. 13/812,324, filed Jan. 25, 2013, U.S. Publication No. 2014/0012287, now U.S. Pat. No. 9,901,410, issued on Feb. 27, 2018, the content of which is incorporated herein by reference in its entirety.

This application is related to U.S. patent application Ser. No. 15/874,189, filed Jan. 18, 2018, U.S. Publication No. 2018-0206923 the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US2012/070924, filed Dec. 20, 2012, PCT Publication No. WO2013/096610, the content of which is incorporated herein by reference in its entirety.

This application is related to U.S. patent application Ser. No. 14/364,195, filed Jun. 10, 2014, U.S. Publication No. 2014/0318299, now U.S. Pat. No. 9,364,955 issued on Jun. 14, 2016, the content of which is incorporated herein by reference in its entirety.

This application is related to U.S. patent application Ser. No. 15/180,503, filed Jun. 13, 2016, U.S. Publication No. 2017/0015007, now U.S. Pat. No. 9,821,477, issued on Nov. 21, 2017, the content of which is incorporated herein by reference in its entirety.

This application is related to U.S. patent application Ser. No. 15/786,901, filed Oct. 18, 2017, U.S. Publication No. 2018/0161992, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US2013/054326, filed Aug. 9, 2013, PCT Publication No. WO2014/026104, the content of which is incorporated herein by reference in its entirety.

This application is related to U.S. patent application Ser. No. 14/418,993, filed Feb. 2, 2015, U.S. Publication No. 2015/0282835, now U.S. Pat. No. 9,675,380 issued on Jun. 13, 2017, the content of which is incorporated herein by reference in its entirety.

This application is related to U.S. patent application Ser. No. 15/619,875, filed Jun. 12, 2017, U.S. Publication No. 2018/0021060, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US2014/010808, filed Jan. 9, 2014, PCT Publication No. WO2014/110218, the content of which is incorporated herein by reference in its entirety.

This application is related to U.S. patent application Ser. No. 14/759,020, filed Jul. 2, 2015, U.S. Publication No. 2015/0342690, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US2013/043858, filed Jun. 3, 2013, PCT Publication No. WO2013/184560, the content of which is incorporated herein by reference in its entirety.

This application is related to U.S. patent application Ser. No. 14/402,224, filed Nov. 19, 2014, U.S. Publication No. 2015/0150633, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US2013/038701, filed May 20, 2014, PCT Publication No. WO2014/189876, the content of which is incorporated herein by reference in its entirety.

This application is related to U.S. patent application Ser. No. 14/888,541, filed Nov. 2, 2015, U.S. Publication No. 2016/0074028, now U.S. Pat. No. 9,517,059, issued on Dec. 13, 2016, the content of which is incorporated herein by reference in its entirety.

This application is related to U.S. patent application Ser. No. 15/350,549, filed Nov. 14, 2016, U.S. Publication No. 2017/0119364, now U.S. Pat. No. 10,016,187, issued on Jul. 10, 2018, the content of which is incorporated herein by reference in its entirety.

This application is related to U.S. patent application Ser. No. 16/020,115, filed Jun. 27, 2018, U.S. Publication No. 2018/0368823, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US2014/036571, filed May 2, 2014, PCT Publication No. WO2014/179683, the content of which is incorporated herein by reference in its entirety.

This application is related to U.S. patent application Ser. No. 14/888,189, filed Oct. 30, 2015, U.S. Publication No. 2016/0067000, now U.S. Pat. No. 9,913,695, issued on Mar. 13, 2018, the content of which is incorporated herein by reference in its entirety.

This application is related to U.S. patent application Ser. No. 15/916,664, filed Mar. 9, 2018, U.S. Publication No. 2018/0256269, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US2014/067091, filed Nov. 24, 2014, PCT Publication No. WO2015/081008, the content of which is incorporated herein by reference in its entirety.

This application is related to U.S. patent application Ser. No. 15/038,531, filed May 23, 2016, U.S. Publication No. 2016/0287224, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US2015/034424, filed Jun. 5, 2015, PCT Publication No. WO2015/188071, the content of which is incorporated herein by reference in its entirety.

This application is related to U.S. patent application Ser. No. 15/315,868, filed Dec. 2, 2016, U.S. Publication No. 2017/0100197, the content of which is incorporated herein by reference in its entirety.

This application is related to U.S. patent application Ser. No. 16/225,156, filed Dec. 19, 2018, U.S. Publication No. 2019/xxxxxx, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US2016/028374, filed Apr. 20, 2016, PCT Publication No. WO2016/172162, the content of which is incorporated herein by reference in its entirety.

This application is related to U.S. patent application Ser. No. 15/567,109, filed Oct. 17, 2017, U.S. Publication No. 2018-0228557 the content of which is incorporated herein by reference in its entirety.

This application is related to U.S. Provisional Application No. 62/401,390, filed Sep. 29, 2016, the content of which is incorporated herein by reference in its entirety.

This application is related to PCT Application No. PCT/US2017/054297, filed Sep. 29, 2017, PCT Publication No. WO2018/064475, the content of which is incorporated herein by reference in its entirety.

This application is related to U.S. Provisional Application No. 62/517,433, filed Jun. 9, 2017, the content of which is incorporated herein by reference in its entirety.

This application is related to PCT Application No. PCT/US2018/036876, filed Jun. 11, 2018, PCT Publication No. WO2018/227180, the content of which is incorporated herein by reference in its entirety.

This application is related to U.S. Provisional Application No. 62/481,309, filed Apr. 4, 2017, the content of which is incorporated herein by reference in its entirety.

This application is related to U.S. Provisional Application No. 62/598,812, filed Dec. 14, 2017, the content of which is incorporated herein by reference in its entirety.

This application is related to U.S. Provisional Application No. 62/617,513, filed Jan. 15, 2018, the content of which is incorporated herein by reference in its entirety.

This application is related to PCT Application No. PCT/US2018/026016, filed Apr. 4, 2018, PCT Publication No. WO2018/187425 the content of which is incorporated herein by reference in its entirety.

This application is related to U.S. Provisional Application No. 62/533,644, filed Jul. 17, 2017, the content of which is incorporated herein by reference in its entirety.

This application is related to U.S. Provisional Application No. 62/614,263, filed Jan. 5, 2018, the content of which is incorporated herein by reference in its entirety.

This application is related to PCT Application No. PCT/US2018/042449, filed Jul. 17, 2018, PCT Publication No. WO2019/xxxxxx, the content of which is incorporated herein by reference in its entirety.

This application is related to U.S. Provisional Application No. 62/582,283, filed Nov. 6, 2017, the content of which is incorporated herein by reference in its entirety.

This application is related to U.S. Provisional Application No. 62/614,346, filed Jan. 5, 2018, the content of which is incorporated herein by reference in its entirety.

This application is related to PCT Application No. PCT/US2018/059338, filed Nov. 6, 2018, PCT Publication No. WO2019/xxxxxx, the content of which is incorporated herein by reference in its entirety.

This application is related to U.S. Design application Ser. No. 29/632,148, filed Jan. 5, 2018, the content of which is incorporated herein by reference in its entirety.

This application is related to U.S. Pat. No. 9,011,318, issued Apr. 21, 2015, the content of which is incorporated herein by reference in its entirety.

### BACKGROUND

As less invasive medical techniques and procedures become more widespread, medical professionals such as surgeons may require articulating surgical tools, such as endoscopes, to perform such less invasive medical techniques and procedures that require access to locations within the patient, such as a site accessible through the mouth or other natural orifice, or a site accessible through an incision through the patient's skin.

There is a need for improved systems for performing a medical procedure.

### SUMMARY

In an aspect, a system for performing a medical procedure on a patient, comprises: an articulating probe assembly, comprising: an inner probe comprising multiple articulating inner links; an outer probe surrounding the inner probe and comprising multiple articulating outer links; and at least two working channels that exit a distal portion of the probe assembly, at least one tool configured to translate through one of the at least two working channels, and a robotic stand for positioning the articulating probe relative to the patient.

In some embodiments, the robotic stand comprises a joint and arm length geometry constructed and arranged for maximal patient access, auxiliary instrumentation and sensors, view-ability of the patient, and co-procedure activity.

In some embodiments, the robotic stand comprises a joint and arm length geometry that is constructed and arranged for operation in compact environments.

In some embodiments, the robotic stand comprises a joint geometry constructed and arranged for coordinated motion to provide access in small operating spaces.

In some embodiments, the robotic stand is constructed and arranged in a compact design for a single person to manipulate.

In some embodiments, the robotic stand comprises a roll axis geometry constructed and arranged for instruments to be twisted about a single location without having to move other joints.

In some embodiments, the robotic stand is constructed and arranged to transition to a compact stowing arrangement.

In some embodiments, a display and control arrangement is shared between the robotic stand and a user interface.

In some embodiments, the robotic stand is constructed and arranged to be maneuverable by a single person.

In some embodiments, the robotic stand comprises four wheels.

In some embodiments, the robotic stand further comprises a braking mechanism.

In some embodiments, the braking mechanism allows the four wheels to be locked with one pedal.

In some embodiments, the robotic stand comprises at least one motor.

In some embodiments, the at least one motor comprises at least one channel.

In some embodiments, the robotic stand comprises a handle.

In some embodiments, the handle is constructed and arranged to allow access for manual push-pull from all sides of the robotic stand for manual positioning thereof.

In some embodiments, the robotic stand is constructed and arranged to perform at least one procedure at any side of a bed.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the preferred embodiments.

FIG. 1 is a schematic view of a system in which embodiments of the present inventive concepts can be practiced.

FIGS. 1A-C are graphic demonstrations of a robotic probe, in accordance with embodiments of the present inventive concepts.

FIG. 2 is a perspective view of a robotic stand, in accordance with embodiments of the present inventive concepts.

FIG. 2A is a closeup perspective view of an operation of the robotic stand of FIG. 2, in accordance with embodiments of the present inventive concepts.

FIG. 2B is a closeup perspective view of the first articulation hub of FIGS. 2 and 2A, in accordance with embodiments of the present inventive concepts.

FIG. 2C is a closeup perspective view of the second articulation hub of FIG. 2, in accordance with embodiments of the present inventive concepts.

FIG. 3 is a perspective view of a motor, in accordance with embodiments of the present inventive concepts.

FIG. 4 is a front view of the tower of the robotic stand of FIG. 2 including a user interface, in accordance with embodiments of the present inventive concepts.

FIG. 5 is a perspective view of the base of the robotic stand of FIG. 2 comprising a recess, in accordance with embodiments of the present inventive concepts.

FIG. 6A is a front view of the robotic stand and feeder of FIGS. 2-5, in accordance with embodiments of the present inventive concepts.

FIG. 6B is a side view of the robotic stand and feeder of FIGS. 2-6A, in accordance with embodiments of the present inventive concepts.

FIG. 6C is a closeup view of the robotic stand and feeder of FIGS. 2-6B in a stowed position, in accordance with embodiments of the present inventive concepts.

FIG. 7 is a side view of the robotic stand and feeder of FIGS. 2-6A in a fully extended or deployed position, in accordance with embodiments of the present inventive concepts.

FIG. 8 is a top view of a surgical table and various positions of the robotic stand relative to the surgical table and potential surgeon locations, in accordance with embodiments of the present inventive concepts.

FIG. 9A is a top view of the robotic stand positioning the feeder above a patient table, in accordance with embodiments of the present inventive concepts.

FIG. 9B is a side view of the robotic stand of FIG. 9A positioning the feeder above the patient table, in accordance with embodiments of the present inventive concepts.

FIGS. 10A-C are views of three different possible positions of the feeder by the robotic stand for different surgical procedures, in accordance with embodiments of the present inventive concepts.

FIG. 11 is a view of the robotic stand in an extended or deployed position, in accordance with embodiments of the present inventive concepts.

FIG. 12 is a view of the robotic stand in a stowed position, in accordance with embodiments of the present inventive concepts.

### DETAILED DESCRIPTION OF EMBODIMENTS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate” shall include locations relatively close to, on, in and/or within a referenced component, anatomical location, or other location.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

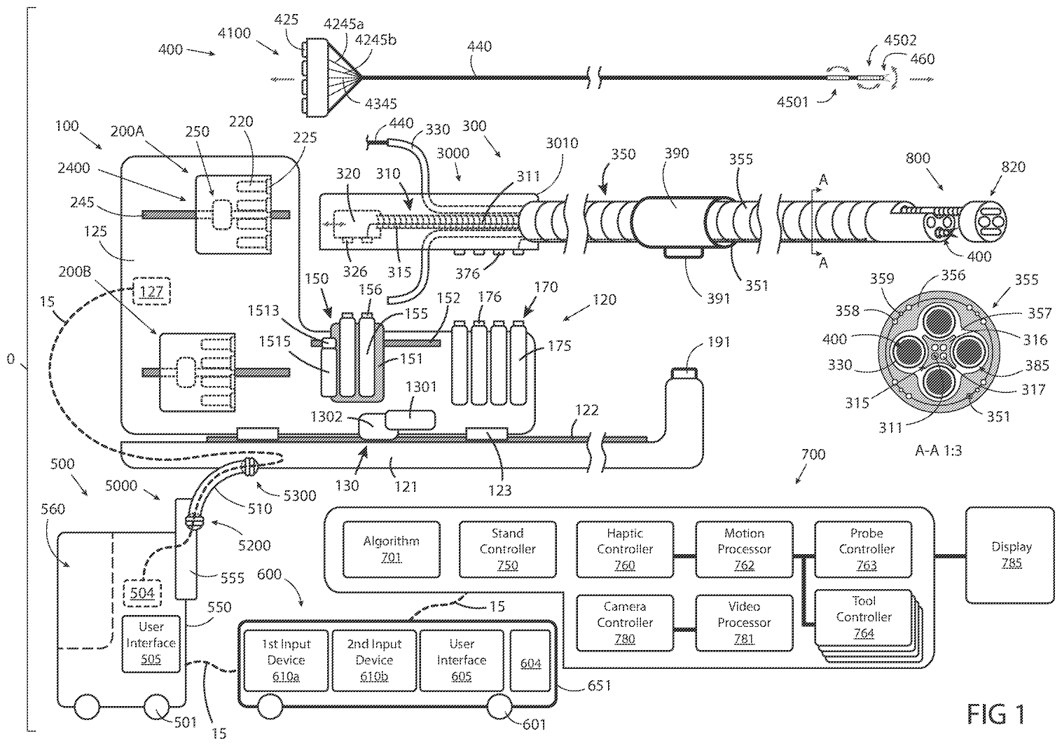

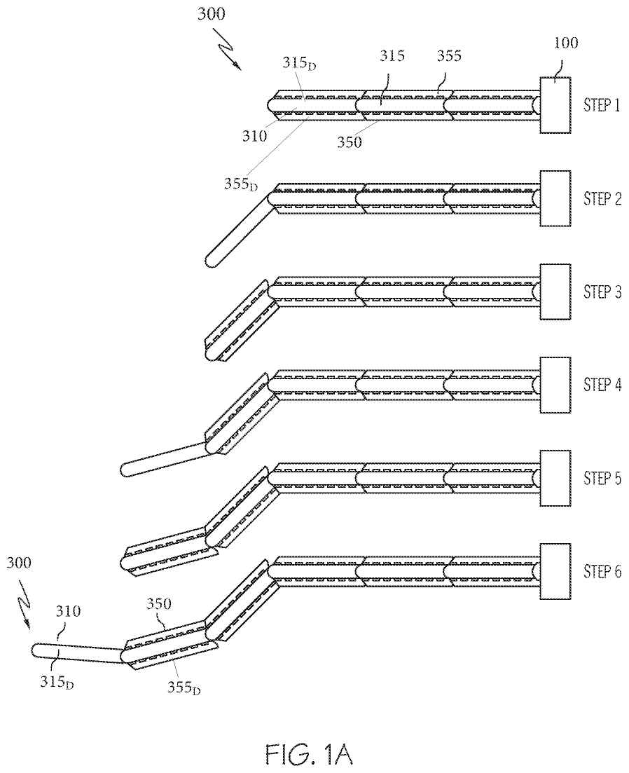

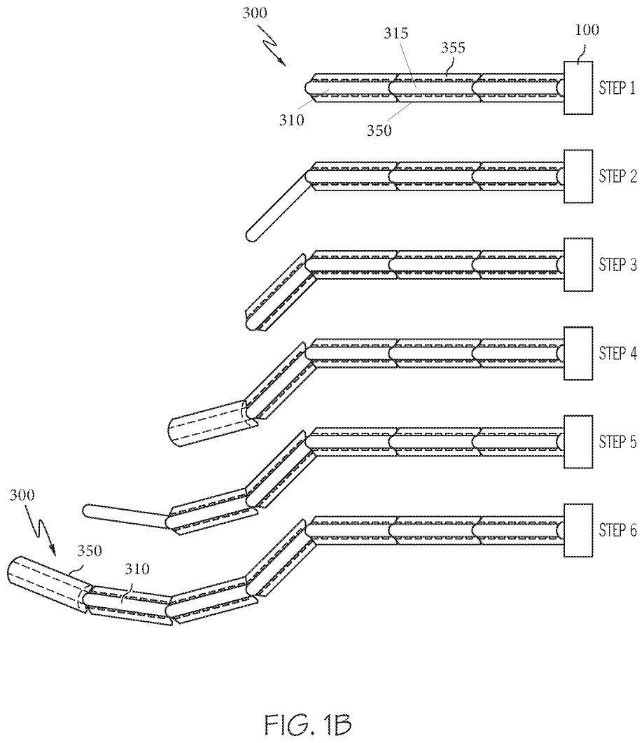

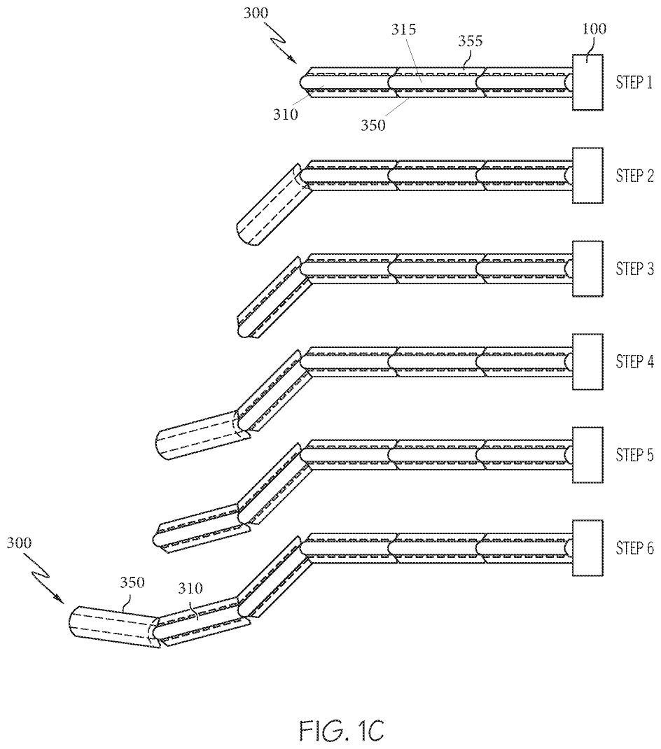

Referring to FIG. 1, a schematic view of a system in which embodiments of the present inventive concepts can be practiced is illustrated.

System 10 includes a robotic feeder 100. Feeder 100 interchangeably and operably engages a robotic probe assembly 300, and at least one robotic tool assembly 400. Feeder 100 is constructed and arranged to advance, retract, steer, and/or otherwise control the position and/or articulation of probe assembly 300 and/or tools 400, as described herein. One or more tools 400 can be slidingly received within a channel of probe assembly 300, and each tool 400 can be advanced beyond the distal end of probe assembly 300. Feeder 100 includes a probe manipulation assembly 120 for operably controlling the position and articulation of probe assembly 300. Feeder 100 also includes at least one tool manipulation assembly, tool drive 200 (e.g. tool drives 200A and 200B shown), for controlling the position and articulation of an attached tool 400. System 10 further includes a robotic positioning assembly, stand 500. Stand 500 includes an articulation assembly 5000 for positioning feeder 100 with multiple degrees of freedom, for example within an operating room, relative to a patient and/or patient bed, as described herein. System 10 further includes a control interface, surgeon console 600, configured to receive commands from one or more operators of system 10 (e.g. one or more surgeons or other clinicians). Console 600 can include a first and second input device, 610A and 610B respectively (singly or collectively input devices 610 herein), each configured to receive robotic input data (e.g. via a kinematic input device as described herein). System 10 further includes a collection of data processing components, collectively processing unit 700. Processing unit 700 can include one or more algorithms, controllers, memory, state machines, and/or processors, singly and/or collectively controlling one or more components of system 10 (e.g. based at least on one or more user inputs received by one or more input components of system 10). System 10 further includes an imaging device, camera assembly 800 (e.g. a tool 400 configured as a camera, as described herein), comprising one or more cameras, camera 820. Image data (e.g. still and/or video images) captured by camera 820 can be displayed on one or more monitors or other screens, display 785. One or more components described herein as included in a tool 400 can also be included in camera assembly 800, for example camera assembly 800 can comprise a tool 400 with camera 820 operably attached thereto. A conduit, bus 15, can connect one or more components of system 10. Bus 15 can comprise one or more electrical, fluid, optical, and/or other conduits for transferring information, power, one or more fluids, light energy, and combinations of one or more of these.

### Probe Assembly 300

Probe assembly 300 includes an outer probe 350, comprising multiple articulating outer links 355. Links 355 each comprise a ring-like structure (e.g. a hollow tube-like structure), link body 356, surrounding a hollow bore, channel 357. Collectively, channels 357 define a lumen extending along at least a portion of the length of outer probe 350. Links 355 can include multiple lumens extending therethrough, such as lumens extending along the link, through link body 356. For example, links 355 can include one or more steering cable lumens, lumens 358, such as eight lumens 358 shown. Lumens 358 can each slidingly receive a steering cable 351 that is used to control at least the articulation of outer probe 350, as described herein. Links 355 can also include one or more auxiliary lumens, four lumens 359 shown. In some embodiments, lumens 359 can slidingly receive elongate devices and/or filaments, such as optical fibers for delivering light to a surgical site.

Probe assembly 300 further includes inner probe 310, comprising multiple articulating inner links 315. Inner probe 310 is slidingly received within channels 356 extending through outer probe 350. Links 315 can comprise a link body 316, and can include multiple lumens extending therethrough, such as lumens extending along the link. For example, links 315 can include one or more steering cable lumens, lumens 317, such as four lumens 318 shown. Lumens 317 can each slidingly receive a steering cable 311 used to control at least the articulation of inner probe 310, as described herein.

The outer shape of link body 316 can align with the shape of the channel 357 to form a plurality of passageways or working channels 385, extending throughout probe assembly 300. Working conduits 330 can be slidingly received within channels 385, extending throughout the probe assembly 300. Each conduit 330 can sliding receive at least a portion of a tool 400.

Probe assembly 300 can be of similar construction and arrangement to the similar device described in reference to applicant's co-pending U.S. patent application Ser. No. 16/114,681, filed Aug. 28, 2018, the content of which is incorporated herein by reference in its entirety.

Probe assembly 300 further comprises a manipulation assembly 3000, operably attached to the proximal portion of probes 310, 350. Manipulation assembly 3000 comprises a housing 3010, surrounding at least a cart 320, operably attached to inner probe 310. Manipulation assembly 3000 comprises one or more bobbins 376 operably attached to one or more steering cables 351 (also referred to herein as control cables). Cart 320 comprises one or more bobbins 326 operably attached to one or more steering cables 311. Manipulation assembly 3000 is constructed and arranged to operably and removably attach to feeder 100, as described herein. Manipulation assembly 3000 supports the proximal sections of one or more working conduits 330 in an orientation that is radially dispersed relative to the radially compact orientation of the distal portions of working conduits 330 within probe assembly 300.

Probe assembly 300 can include a support structure, introducer 390. Introducer 390 can comprise a rigid elongate structure. Introducer 390 can surround at least a portion of probe assembly 300. Introducer 390 can comprise a connector portion 391, constructed and arranged to operably attach to a portion of feeder 100 as described herebelow.

Probe assembly 300 can be of similar construction and arrangement to the similar device described in applicant's co-pending application U.S. Provisional Application No. 62/614,240, filed Jan. 5, 2018, the content of which is incorporated herein by reference in its entirety.

### Feeder 100

Feeder 100 comprises a manipulation assembly 120 comprising a carriage 125 operably attached to a base 121. Carriage 125 can comprise one or more linear bearings 123 fixedly attached thereto, slidingly attached to a linear rail assembly 122, which in turn is fixedly attached to base 121. Linear rail assembly 122 can comprise one or more rails and/or lead screws. Manipulation assembly 120 can comprise a linear drive assembly 130, that is operably attached to carriage 125 and linear rail assembly 122. For example, linear rail assembly 122 can comprise at least a lead screw, and linear drive assembly 130 can comprise a motor 1301 and gear box 1302. Linear drive assembly 130 can be configured to engage the lead screw of linear rail assembly 122, such as to translate carriage 125 relative to base 121.

Manipulation assembly 120 can comprise a probe support assembly 170. Probe support assembly 170 can comprise at least a portion of carriage 125. Probe support assembly 170 can comprise one or more motors 175, each operably attached to a capstan 176. Probe support assembly 170 is constructed and arranged to operably and removably attach to manipulation assembly 3000, for example, such that each capstan 176 operably engages a corresponding bobbin 376. Motors 175 can be configured to rotate capstans 176, which in turn rotate bobbins 376, tensioning and de-tensioning cables 351 to control the articulation of outer probe 350.

Probe support assembly 170 can further comprise a probe translation assembly 150. Probe translation assembly 150 can comprise one or more motors 155, each operably attached to a capstan 156. Probe translation assembly 150 is constructed and arranged to operably and removably attach to cart 320, for example such that each capstan 156 operably engages a corresponding bobbin 326. Motors 155 can be configured to rotate capstans 156, which in turn rotate bobbins 326, tensioning and de-tensioning cables 311 to control the articulation of inner probe 310. Probe translation assembly 150 can comprise a cart 151. Motors 155 can be fixedly attached to cart 151. Cart 151 can be slidingly attached to a linear rail assembly 152, fixedly attached to carriage 125. Linear rail assembly 152 can comprise one or more rails and/or lead screws. Probe translation assembly 150 can comprise a motor 1515 and drive gear 1513 operably attached thereto. Drive gear 1513 can operably attach to linear rail assembly 152, for example when linear rail assembly 152 comprises at least a lead screw. Motor 1515 can be configured to rotate drive gear 1513 to translate cart 151 relative to carriage 125. Cart 151 can be constructed and arranged to engage cart 320, such that translation of cart 151 causes the translation of cart 320 within manipulation assembly 3000. Translation of cart 320 can cause the translation of inner probe 310 with respect to outer probe 350, as described herein.

Feeder 100 can include a connector portion 191, constructed and arranged to removably connect to introducer 390 of probe assembly 300. Connector portion 191 can be positioned at the distal end of carriage 125, as shown.

Feeder 100 can include one or more modules 127, such as one or more processors and/or controllers. Module 127 can be operably attached to one or more components of system 10 via bus 15.

Feeder 100 can be of similar construction and arrangement to the similar device described in applicant's co-pending application U.S. Provisional Application No. 62/614,240, filed Jan. 5, 2018, the content of which is incorporated herein by reference in its entirety.

### Tool Drive 200

Each tool drive 200 (also referred to herein as a singular tool drive 200) is configured to operably and interchangeably attach to one or more tools 400. Feeder 100 can comprise one, two, three, four, or more tool drives, tool drives 200A and 200B shown. Additional tool drives can be mounted to carriage 125 opposite tool drives 200A and 200B (e.g. on the opposite side of carriage 125). Tool drive 200 can slidingly attach to carriage 125 via a translation assembly 2400. Translation assembly 2400 can comprise a linear rail assembly 245, fixedly attached to carriage 125. Linear rail assembly 245 can comprise one or more rails and/or lead screws. Translation assembly 2400 can further comprise a linear drive assembly 250, operably attached to tool drive 200 and linear rail assembly 245. For example, linear rail assembly 245 can comprise at least a lead screw, and linear drive assembly 250 can comprise a motor and/or a gear box. Linear drive assembly 250 can be configured to engage the lead screw of linear rail assembly 245, to translate tool drive 200 relative to carriage 125. Translation of tool drive 200 can cause the translation of an attached tool 400, for example relative to outer probe 350 operably attached to manipulation assembly 120.

Tool drive 200 can comprise one or more motors 220, configured to manipulate one or more components of tool drive 200. For example, one or more motors 220 can be configured to rotate one or more assemblies of tool drive 200 relative to each other, and/or to rotate one or more gears 225 (e.g. capstans) of tool drive 200. Gears 225 of tool drive 200 can be configured to operably engage one or more bobbins of an attached tool 400, as described herein, to control the articulation of the attached tool 400.

Tool drive 200 can be of similar construction and arrangement to the similar device described in applicant's co-pending application U.S. Provisional Application No. 62/614,228, filed Jan. 5, 2018, the content of which is incorporated herein by reference in its entirety.

### Tool 400

Tool 400 can include a manipulation assembly 4100, operably attached to the proximal end of a shaft 440. Shaft 440 can comprise a flexible shaft, comprising one or more lumens. Tool 400 can comprise one or more sets of steering (or control) cables 4245a, 4245b, and or 4345. Cables 4245a,b can be operably attached to manipulation assembly 4100, and extend through shaft 440 to a first and second articulation section 4501 and 4502, respectively. Cables 4245a,b can be tensioned and/or de-tensioned by manipulation assembly 4100 to cause the articulation of articulation sections 4501 and 4502, respectively. Cables 4345 can be operably attached to manipulation assembly 4100, and extend through shaft 440 to an end effector 460. Cables 4345 can be tensioned and/or de-tensioned by manipulation assembly 4100 to cause the articulation or other manipulation of end effector 460. System 10 can comprise multiple tools 400, such as four, five, six, or more tools 400, each exchangeable and operably attachable to tool drives 200. End effectors 460 can comprise scissors, graspers, blades, cautery devices, laser devices, and the like. Manipulation assembly 4100 can be constructed and arranged to removably attach to tool drive 200, such that gears 225 engage bobbins 425 of manipulation assembly 4100. Motors 220 of tool drive 200 can rotate gears 225, and bobbins 425, to tension and/or de-tension one or more cables of tool 400 described herein, to tension and/or de-tension the cables and manipulate tool 400. Manipulation assembly 4100 can also be constructed and arranged to rotate one or more components of tool 400 relative to each other, for example to rotate end effector 460 relative to shaft 440.

Tool 400 can be of similar construction and arrangement to the similar device described in applicant's co-pending application U.S. Provisional Application No. 62/614,225, filed Jan. 5, 2018, the content of which is incorporated herein by reference in its entirety.

### Camera Assembly 800

In some embodiments, as described hereabove, a tool 400 can be configured as a camera assembly 800. Camera assembly 800 can comprise a camera 820, operably attached to the distal end of shaft 440 of a tool 400. In some embodiments, camera 820 is attached to shaft 440 after shaft 440 has been inserted through probe assembly 300. For example, in some embodiments, camera 820 is larger than working channel 385.

Camera assembly 800 can be of similar construction and arrangement to the similar device described in applicant's co-pending application PCT International Patent Application No. PCT/US2018/059338, filed Nov. 6, 2018, the content of which is incorporated herein by reference in its entirety.

### Stand 500

Stand 500 can be constructed and arranged to position feeder 100 relative to a patient and/or patient bed, such as to position probe assembly 300 for a surgical procedure. For example, surgical procedures can include but are not limited to transabdominal procedures, transoral procedures, trans anal procedures, and/or trans vaginal procedures. Stand 500 includes a base 550, supporting an articulation assembly 5000. Articulation assembly 5000 includes a tower 555, extending vertically from base 550. A first hub 5200 is operably attached to tower 555. First hub 5200 can be adjusted along the height of tower 555, via one or more motors and/or vertical translation assemblies. First hub 5200 is operably attached to positioning arm 510, which is operably attached to a second hub 5300. Second hub 5300 is operably attached to base 121 of feeder 100. Hubs 5200 and 5300 can each comprise one or more motors, gears, hinges, axles, and the like, configured to manipulate the position of feeder 100 relative to stand 500. Bus 15 of system 10 can operably connect feeder 100 to stand 500. In some embodiments, bus 15 is routed through hubs 5200, 5300, arm 510, and/or tower 555, such that bus 15 is at least partially contained within articulation assembly 5000.

Stand 500 can comprise a recess 560. Articulation assembly 5000 can be configured to “fold” into a stowed position, with feeder 100 positioned at least partially within recess 560. In some embodiments stand 500 can comprise a processor 504 and a user interface 505. User interface 505 can include input and output functionality, such as a touchscreen monitor. User interface 505 can be configured to allow a user to control one or more components of system 10, for example the articulation of articulation assembly 5000. In some embodiments, stand 500 includes one or more wheels 501, and is constructed and arranged to be mobile. For example, stand 500 can be manually repositionable by a user and/or can be robotically repositionable, for example when wheels 501 are driven by one or more motors.

Stand 500 can be of similar construction and arrangement to the similar device described in applicant's co-pending application U.S. Provisional Application No. 62/614,223, filed Jan. 5, 2018, the content of which is incorporated herein by reference in its entirety.

### Surgeon Console 600

Surgeon console 600 can be operably attached to one or more components of system 10, such as via bus 15. Console 600 can comprise a base 651, supporting input devices 610a,b, and user interface 605. Console 600 can comprise a processor 604. In some embodiments, processor 604 can receive commands from input device 610a,b, and/or user interface 605. User interface 605 can be configured to allow a user to control one or more components of system 10. In some embodiments, user interface 605 can be a redundant interface of user interface 505, such that a user can perform the same operations from either interface. In some embodiments, console 600 includes one or more wheels 601, and is constructed and arranged to be mobile. For example, console 600 can be manually repositionable by a user and/or can be robotically repositionable, for example when wheels 601 are driven by one or more motors.

Console 600 can be of similar construction and arrangement to the similar device described in applicant's co-pending application U.S. Provisional Application No. 62/614,224, filed Jan. 5, 2018, the content of which is incorporated herein by reference in its entirety.

### Processor 700

Processing unit 700 can comprise one or more controllers and/or processors, located throughout system 10. For example, processor 700 can comprise a computer or other processing device, and/or can comprise one or more controllers or modules of system 10 (e.g. module 127 of feeder 100, processor 504 of stand 500, and/or processor 604 of user interface 600). Processing unit 700 can comprise one or more algorithms for processing data and/or commanding one or more components of system 10 to perform one or more operations. Processing unit 700 can comprise one or more controllers for controlling components of system 10. Processing unit 700 can comprise a stand controller 750, for operational control of stand 500. Processing unit 700 can comprise a camera controller, for operational control of camera assembly 800. Camera controller 780 can be operably attached to a video processor 781 for processing image data captured by camera 820. Video processor 781 can provide processed image data to a display 785, for display to a user. Processing unit 700 can comprise a haptic controller 760, operably attached to input devices 610a,b of console 600, for example via processor 604. Haptic controller 760 can be operably attached to a motion processor 762, which is operably attached to a probe controller 763, and one or more tool controllers 764. Haptic controller 760 can receive robotic input data (e.g. via a kinematic input device) from input devices 610a,b, and/or provide haptic feedback commands to input devices 610a,b. Motion processor 762 can process the robotic input data, and provide articulation and/or translation commands to probe controller 763 and/or tool controllers 764. Probe controller 763 can provide commands to one or more motors of system 10, for example to one or more motors of manipulation assembly 120 to at least advance, retract, steer, and/or otherwise control the position and/or articulation of probe assembly 300. Tool controllers 764 can provide commands to one or more motors of system 10, for example one or more motors of a tool drive 200 to at least advance, retract, steer, and/or otherwise control the position and/or articulation of an attached tool 400.

Processor 700 can be of similar construction and arrangement to the similar device described in applicant's co-pending application U.S. Provisional Application No. 62/614,235, filed Jan. 5, 2018, the content of which is incorporated herein by reference in its entirety.

Referring additionally to FIGS. 1A-C, graphic demonstrations of a robotic probe 300 are illustrated, consistent with the present inventive concepts. Articulating probe 300 comprises essentially two concentric mechanisms, an outer mechanism and an inner mechanism, each of which can be viewed as a steerable mechanism. Each of the components of probe 300 can comprise one or more sealing elements, such as to support an insufflation procedure. FIGS. 1A-C show the concept of how different embodiments of robotic probe 300 operate. Referring to FIG. 1A, the inner mechanism can be referred to as a first mechanism or inner probe 310. The outer mechanism can be referred to as a second mechanism or outer probe 350. Each mechanism can alternate between rigid and limp states. In the rigid mode or state, the mechanism is just that—rigid. In the limp mode or state, the mechanism is highly flexible and thus either assumes the shape of its surroundings or can be re-shaped. It should be noted that the term “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume outer probe 350 is rigid and inner probe 310 is limp, as seen in step 1 in FIG. 1A. Now, inner probe 310 is both pushed forward by feeder 100, and a distal-most inner link 315D is steered, as seen in step 2 in FIG. 1A. Now, inner probe 310 is made rigid and outer probe 350 is made limp. Outer probe 350 is then pushed forward until a distal-most outer link 355D catches up to the distal-most inner link 315D (e.g. outer probe 350 is coextensive with inner probe 310), as seen in step 3 in FIG. 1A. Now, outer probe 350 is made rigid, inner probe 310 limp, and the procedure then repeats. One variation of this approach is to have outer probe 350 be steerable as well. The operation of such a device is illustrated in FIG. 1B. In FIG. 1B it is seen that each mechanism is capable of catching up to the other and then advancing one link beyond. According to one embodiment, outer probe 350 is steerable and inner probe 310 is not. The operation of such a device is shown in FIG. 1C.

In medical applications, operation, procedures, and so on, once robotic probe 300 arrives at a desired location, the operator, such as a surgeon, can slide one or more tools through one or more working channels of outer probe 350, inner probe 310, or one or more working channels formed between outer probe 350 and inner probe 310, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of robotic probe 300, such as working channels comprising one or more radial projections extending from outer probe 350, these projections including one or more holes sized to slidingly receive one or more tools. As described with reference to other embodiments, working channels may be positioned on other locations extending from, on, in, and/or within robotic probe 300.

Inner probe 310 and/or outer probe 350 are steerable and inner probe 310 and outer probe 350 can each be made both rigid and limp, allowing robotic probe 300 to drive anywhere in three-dimensions while being self-supporting. Articulating probe 300 can “remember” each of its previous configurations and for this reason, robotic probe 300 can retract from and/or retrace to anywhere in a three-dimensional volume such as the intracavity spaces in the body of a patient such as a human patient.

Inner probe 310 and outer probe 350 each include a series of links, i.e. inner links 315 and outer links 355 respectively, that articulate relative to each other. In some embodiments, outer links 355 are used to steer and lock robotic probe 300, while inner links 315 are used to lock robotic probe 300. In a “follow the leader” fashion, while inner links 315 are locked, outer links 355 are advanced beyond the distal-most inner link 315D. Outer links 355 are steered into position by the system steering cables, and then locked by locking the steering cables. The cable of inner links 315 is then released and inner links 315 are advanced to follow outer links 355. The procedure progresses in this manner until a desired position and orientation are achieved. The combined inner links 315 and outer links 355 may include working channels for temporary or permanent insertion of tools at the surgery site. In some embodiments, the tools can advance with the links during positioning of robotic probe 300. In some embodiments, the tools can be inserted through the links following positioning of robotic probe 300.

One or more outer links 355 can be advanced beyond the distal-most inner link 315D prior to the initiation of an operator controlled steering maneuver, such that the quantity extending beyond the distal-most inner link 315D will collectively articulate based on steering commands. Multiple link steering can be used to reduce procedure time, such as when the specificity of single link steering is not required. In some embodiments, between 2 and 20 outer links can be selected for simultaneous steering, such as between 2 and 10 outer links or between 2 and 7 outer links. The number of links used to steer corresponds to achievable steering paths, with smaller numbers enabling more specificity of curvature of robotic probe 300. In some embodiments, an operator can select the number of links used for steering (e.g. to select between 1 and 10 links to be advanced prior to each steering maneuver).

In some embodiments, the robotic stand 500 comprises a joint and arm length geometry that allows for maximal patient access (e.g. space above the patient), for auxiliary instrumentation and sensors, view-ability of the patient, and co-procedure activity.

In some embodiments, the robotic stand 500 comprises a joint and arm length geometry that allows for operation in low ceiling or compact environments. In some embodiments, the robotic stand 500 comprises a joint geometry that allows for coordinated motion to provide access in small operating spaces.

In some embodiments, the robotic stand 500 comprises a compact design that allows a single person to move, manipulate, and position the robotic stand.

In some embodiments, the robotic stand 500 comprises a roll axis geometry that allows instruments to be twisted about a single location without having to move other joints.

In some embodiments, the robotic stand 500 allows for a compact stowing arrangement, of which there are various geometries. The compact stowing arrangement assists in storage, transport, and deployment. A stow architecture allows for unfolding of the stand directly at bedside. In addition, the architecture allows for unfolding and folding while accommodating a minimal height deployment, or minimal width deployment

In some embodiments, the display and control arrangement is shared between the user interface 505 and the robotic stand 500, his allows for purposely partitioned functions (e.g. joint control only at the robotic stand) or combined functionality (both the user interface 505 and the robotic stand 500 are able to control the joints or the instruments), his also allows for the optimization of functions between sterile and non-sterile personnel.

In some embodiments, the robotic stand 500 is not a fixture and is designed to be maneuverable by a single person.

In some embodiments, the wheel lock 554 allows four wheels 501 to be locked with a single pedal.

In some embodiments, the motor choice and design allows wiring to be conducted through the motors themselves, allowing for a more compact cable routing, thereby minimizing the size of the robotic stand

In some embodiments, the handle 556 allows access to push-pull areas from all sides of the robotic stand, which simplifies positioning and robustness.

In some embodiments, the robotic stand 500 is configurable to multiple procedures from either side of the bed or the end of the bed.

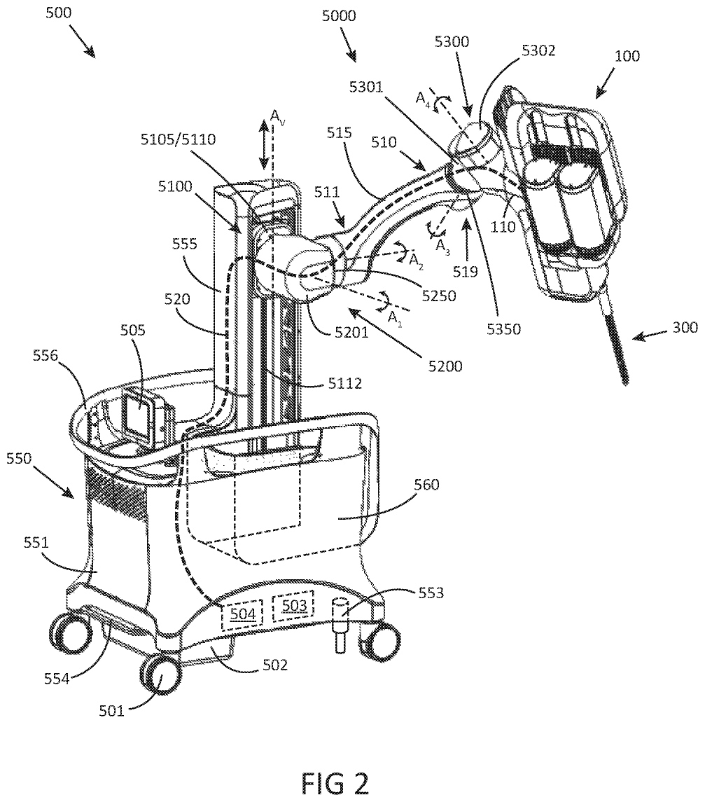

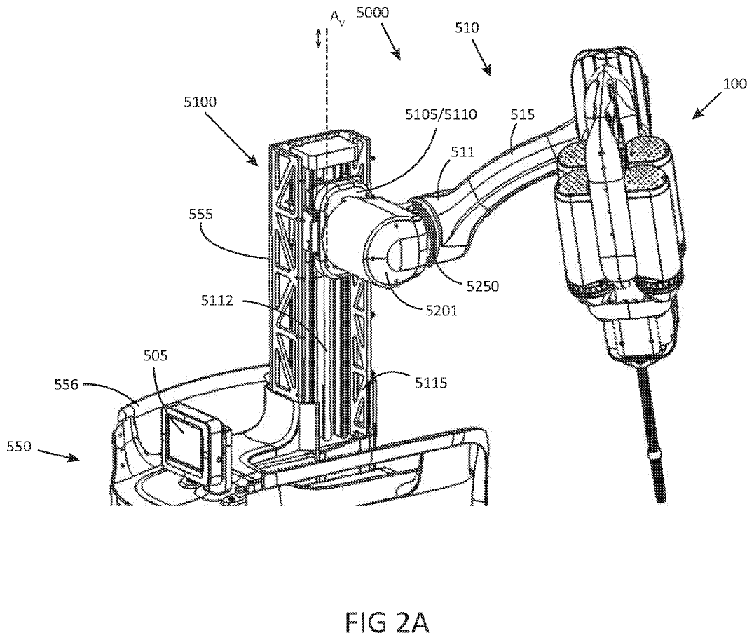

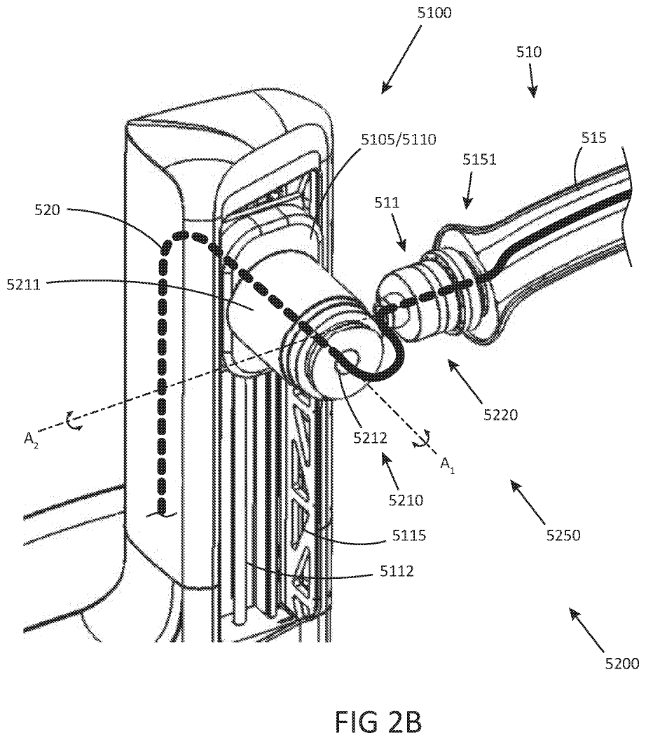

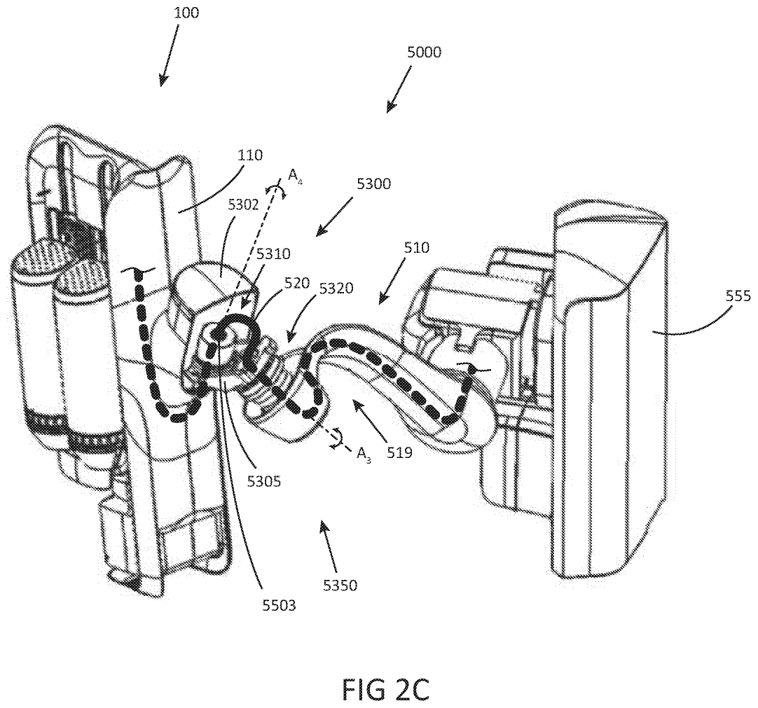

Referring to FIG. 2, a perspective view of a robotic stand 500 is illustrated, in accordance with embodiments of the present inventive concepts.

In some embodiments, the robotic stand 500 includes a base 550. In some embodiments, the base 550 includes a tower 555 that is constructed and arranged to extend vertically from the base 550.

In some embodiments, the robotic stand 500 further includes an articulation assembly 5000 for positioning a feeder 100 relative to the robotic stand 500.

In some embodiments, the base 550 includes a housing 551 that at least partially surrounds a ballast 502, a power supply 503, and a processor 504.

In some embodiments, the base 550 further includes at least one wheel 501 and a wheel lock 554. In some embodiments, the wheel lock 554 includes a caster lock. The caster lock can be depressed (e.g. with a user's foot) to lock the at least one wheel 501. In some embodiments, the wheel lock 554 is coupled to the at least one wheel 501 such that when a user depresses the wheel lock 554 the at least one wheel 501 locks. The wheel 501 may lock via a linkage or the like between wheel lock 554 and wheels 501. In other embodiments, the at least one wheel 501 is locked via a computer controlled locking mechanism.

In some embodiments, the base 550 includes one or more stabilizers 553. In some embodiments, the one or more stabilizers 553 include a piston that extends to contact the ground. In some embodiments, the one or more stabilizers 553 comprise multiple pistons that can lift the robotic stand 500 off of the at least one wheel 501 in order to improve the stability of the robotic stand 500.

In some embodiments, the base 550 further includes a handle 556 for manually positioning the robotic stand 500.

In some embodiments, the robotic stand 500 includes a display having a user interface 505 for controlling the articulation of the articulation assembly 5000 and/or the position of the robotic stand 500, for example, the robotic positioning of robotic stand 500.

In some embodiments, the articulation assembly 5000 includes a vertical positioning assembly 5100, a first articulation hub 5200, and a second articulation hub 5300.

In some embodiments, the articulation assembly 5000 includes a positioning arm 510. In some embodiments, the positioning arm 510 comprises a proximal portion 511 and distal portion 519.

In some embodiments, the positioning arm 510 further comprises a medial portion 515 with a curvilinear profile. In some embodiments, the medial portion 515 may be positioned between the first articulation hub 5200 and the second articulation hub 5300. In some embodiments, the medial portion 515 positions the second articulation hub 5300 away from the first articulation hub 5200. In some embodiments, axis A₂ is aligned with proximal portion 511, as shown in FIG. 2. In some embodiments, axis A₃ is aligned with distal portion 519, as shown in FIG. 2. In some embodiments, medial portion 515 is constructed and arranged in an arc such that axis A₂ is approximately perpendicular to axis A₃ (approximately 90°), but not limited thereto.

In some embodiments, the vertical positioning assembly 5100, the first articulation hub 5200, the second articulation hub 5300, and the positioning arm 510 position the feeder 100 with five degrees of freedom relative to the tower 555, but not limited thereto.

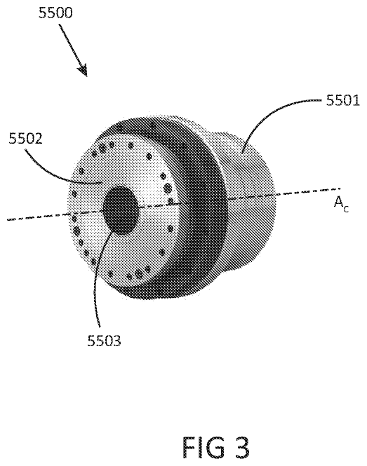

In some embodiments, the first articulation hub 5200 and the second articulation hub 5300 each include one or more motors 5500 (for example, shown and described in FIG. 3). In some embodiments, each motor 5500 includes a central lumen for passing one or more conduits 520.

Referring now to FIG. 2A, a closeup perspective view of an operation of a robotic stand 500 is illustrated, in accordance with embodiments of the present inventive concepts. In some embodiments, the vertical positioning assembly 5100 is positioned within the tower 555 and is constructed and arranged to adjust the height of the positioning arm 510 along axis A_{V}.

In some embodiments, the vertical positioning assembly 5100 includes a sled 5105, operably attached to a motor 5110. In some embodiments, the motor 5110 is configured to drive the sled 5105 vertically along a rail 5112 within the tower 555. The rail 5112 can comprise one or more linear gears, linear bearings, and/or guide rails, and/or other related mechanical components.

In some embodiments, the tower 555 includes a support frame 5115 that is constructed and arranged to provide structural support.

Referring now to FIG. 2B, a closeup perspective view of a first articulation hub 5200 of a robotic stand 500 is illustrated, in accordance with embodiments of the present inventive concepts. In some embodiments, the first articulation hub 5200 includes a casing 5201 (removed for illustrated clarity, but shown in FIG. 2) surrounding a first motor 5210. In some embodiments, the casing 5201 rotatably attaches to sled 5105. In some embodiments, the casing 5201 may be constructed and arranged to rotate about axis A₁, and the rotation may be controlled by motor 5210.

In some embodiments, the casing 5201 rotatably mates with proximal portion 511 of positioning arm 510 to form rotary joint 5250. In some embodiments, the casing 5201 (removed for illustrated clarity, but shown in FIG. 2) surrounds a second motor 5220. The second motor 5220 may control the rotation of positioning arm 510 about axis A₂ relative to the casing 5201.

Referring now to FIG. 2C, a closeup perspective view of a second articulation hub 5300 of a robotic stand 500 is illustrated, in accordance with embodiments of the present inventive concepts. In some embodiments, the second articulation hub 5300 includes a first casing 5301 (removed for illustrated clarity, but shown in FIG. 2). The first casing 5301 rotatably mates with the distal portion 519 of the positioning arm 510 to form a rotary joint 5350. The first casing 5301 may surround a third motor 5320. The third motor 5320 controls the rotation of first casing 5301 about axis A₃ relative to the positioning arm 510.

In some embodiments, the second articulation hub 5300 includes a bracket 5305, fixedly attached to the third motor 5320 and the first casing 5301, such that the bracket 5305 rotates with the first casing 5301 relative to the positioning arm 510.

In some embodiments, a fourth motor 5310 is attached to the bracket 5305 and a base 121 of the feeder 100, such that the feeder 100 rotates relative to axis A₄ and relative to the first casing 5301. In some embodiments, a second casing 5302 (shown) surrounds a portion of the fourth motor 5310 and the bracket 5305, and rotatably engages the first casing 5301.

In some embodiments, the robotic stand 500 comprises one or more conduits 520 for transmitting power and/or data from the base 550, through the articulation assembly 5000, to the feeder 100. The one or more conduits 520 can pass through the central lumens 5503 of the first motor 5210, the second motor 5220, the third motor 5320, and the fourth motor 5310, and the conduits 520 may be fully contained within the tower 555, the casing 5201, the positioning arm 510, the first casing 5301, and/or the second casing 5302, such that conduit 520 is fully contained within the robotic stand 500.

Referring to FIG. 3, a perspective view of a motor 5500 including a central lumen 5503 is illustrated, in accordance with embodiments of the present inventive concepts. Motor 5500 can comprise an outer casing 5501 that surrounds an inner spindle 5502. Inner spindle 5502 can rotate relative to outer casing 5501. Motor 5500 can further comprise a central lumen 5503 that slidingly receives one or more conduits 520 along axis A_{C}, as described hereabove in reference to FIG. 2C. The motor 5500 shown in FIG. 3 may equally apply to motors 5210, 5220, 5310, and/or 5320.

In some embodiments, motor 5500 comprises Harmonic Drive motors (http://www.harmonicdrive.net) incorporated by reference in its entirety, or other motor/gear assemblies that provide a central lumen 5503.

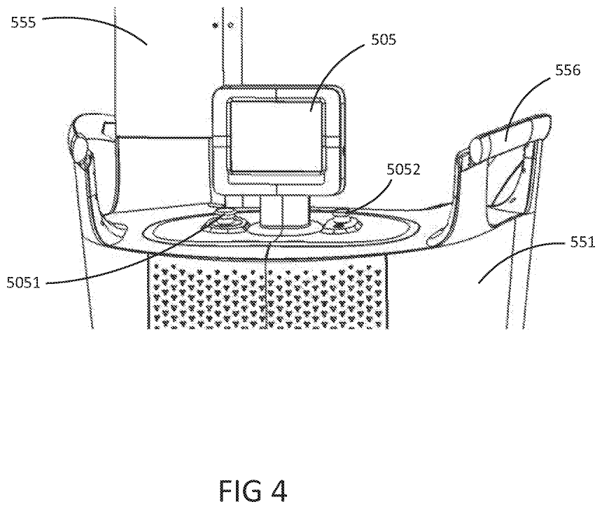

Referring to FIG. 4, a perspective view of a user interface 505 of a robotic stand 500 is illustrated, in accordance with embodiments of the present inventive concepts. User interface 505 can comprise a display and one or more user input devices, such as button 5051 and controller 5052 (e.g. a joystick).

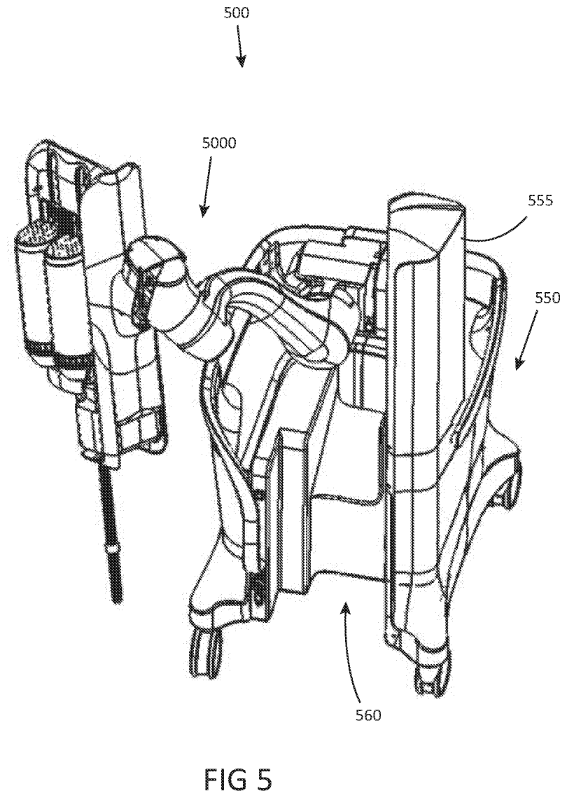

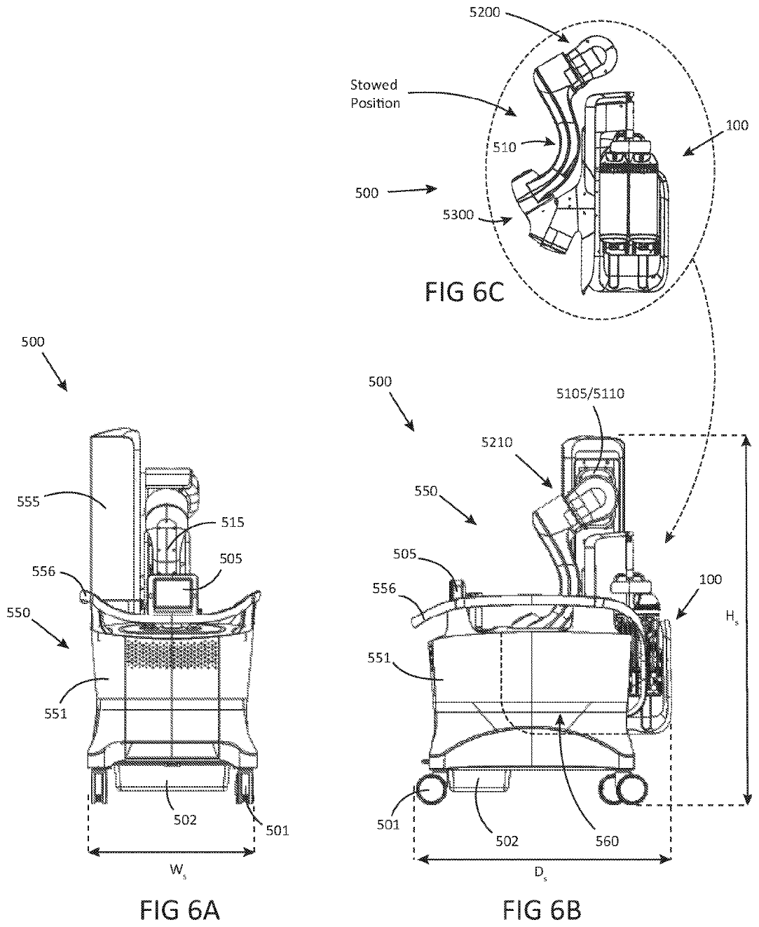

Referring to FIG. 5, a perspective view of a robotic stand 500 with a recess 560 is illustrated, in accordance with embodiments of the present inventive concepts. Robotic stand 500 can comprise a base 550 with a recess 560. Recess 560 can be constructed and arranged such that the articulation assembly 5000 can position the feeder 100 within the recess 560 as shown in FIGS. 6A-C, minimizing the overall volume of the robotic stand 500 and the feeder 100 when in a stowed position.

Referring to FIGS. 6A-6C, front, side, and closeup views of a robotic stand 500 in a stowed position are illustrated, in accordance with embodiments of the present inventive concepts. Also illustrated is the feeder 100 positioned within the recess 560. FIG. 6C in particular shows the relative positions and articulations of the positioning arm 510, the first articulation hub 5200, the second articulation hub 5300, and the feeder 100 in the stowed position. In the stowed position, the robotic stand 500 can comprise a width W_{S}<35 inches, a depth D_{S}<54 inches, and/or a height H_{S}<75 inches.

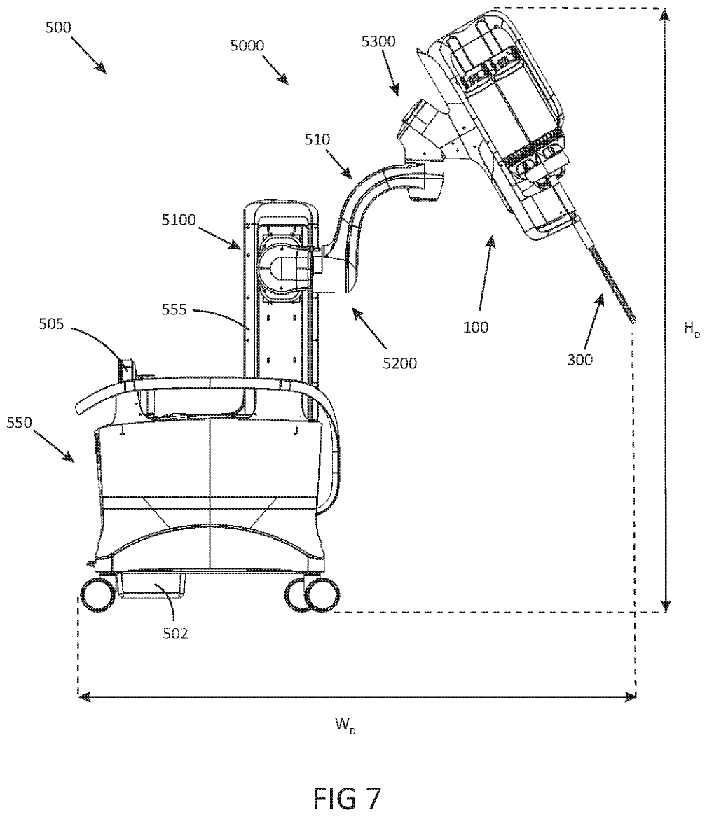

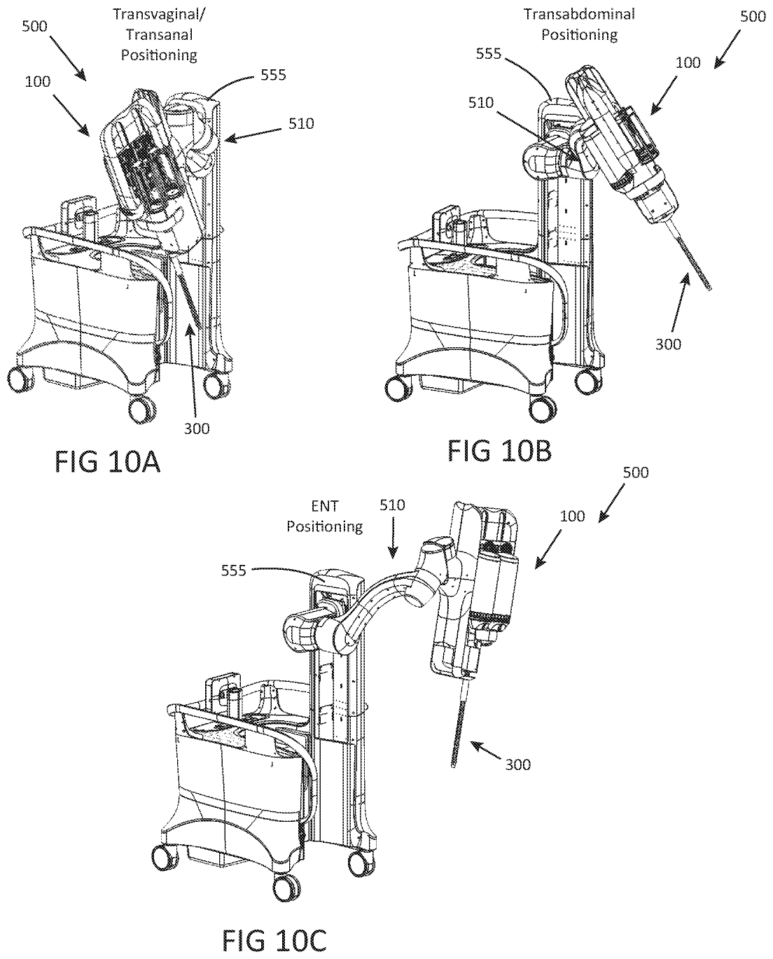

Referring to FIG. 7, a side view of a robotic stand 500 in an extended or deployed position is illustrated, in accordance with embodiments of the present inventive concepts. In the extended or deployed position, the robotic stand 500 can comprise a width W_{D}>95 inches and/or a height H_{D}>105 inches. Also shown are relative positions and articulations of articulation assembly 5000 components in a deployed position. In some embodiments, and in some positions, the overall height and/or width of robotic stand 500 when deployed can be less than W_{D} and/or H_{D}, for example the positions shown in FIGS. 10A-C described herebelow.

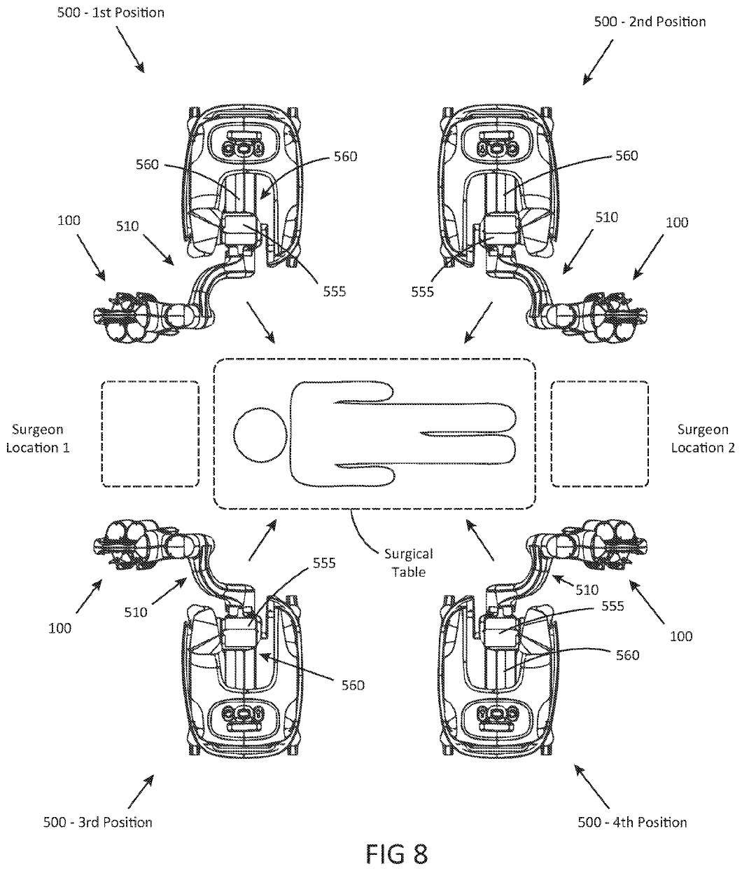

Referring to FIG. 8, a top view of a surgical table and various positions of a robotic stand 500 relative to the surgical table and potential surgeon locations is illustrated, in accordance with embodiments of the present inventive concepts. The robotic stand 500 is therefore constructed and arranged for working on different patient locations, e.g., ENT (ear, nose, throat), colorectal, transabdominal, transvaginal, transanal, and so on. The articulation assembly 5000 can be used to position the feeder 100 in numerous different configurations, as shown.

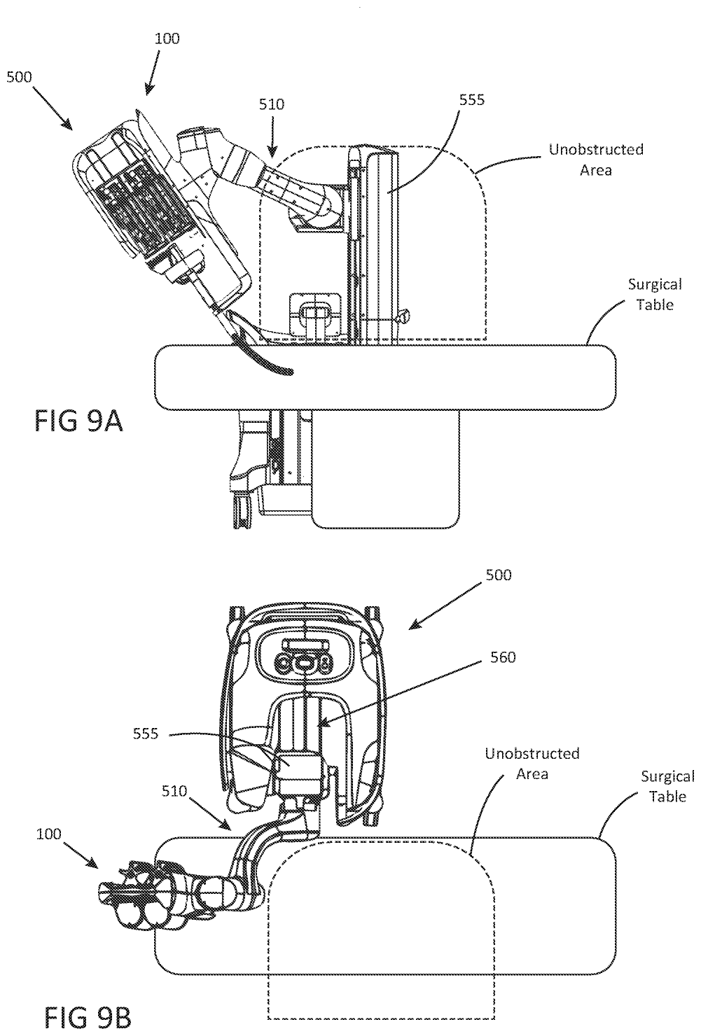

Referring to FIGS. 9A and 9B, side and top views, respectively, of a robotic stand 500 positioning the feeder 100 above a patient table is illustrated, in accordance with embodiments of the present inventive concepts. As also shown, an unobstructed area is enabled by the shape of the arm 510, which is curved to maximize the unobstructed area above the patient to allow for unobstructed access to the patient (e.g. access to the patient is not hindered by the presence of articulation assembly 5000).

Referring to FIGS. 10A-C, three different positions of a feeder 100 by a robotic stand 500 for different surgical procedures are illustrated, in accordance with embodiments of the present inventive concepts. As shown in FIG. 10A, feeder 100 is positioned to accommodate a transvaginal and/or transanal surgical procedure. As shown in FIG. 10B, feeder 100 is positioned to accommodate a transabdominal surgical procedure. As shown in FIG. 10C, feeder 100 is positioned to accommodate an ENT surgical procedure.

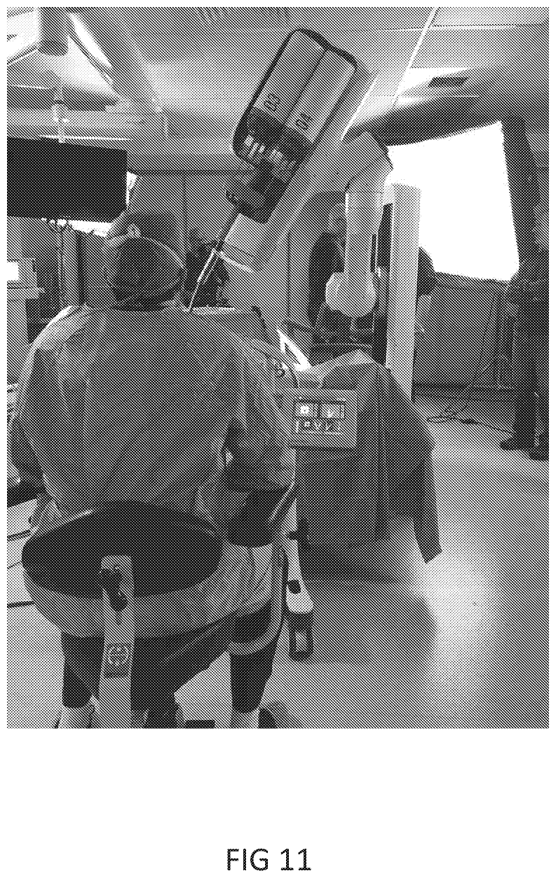

Referring to FIG. 11, a view of the robotic stand 500 in an extended or deployed position is shown, in accordance with embodiments of the present inventive concepts.

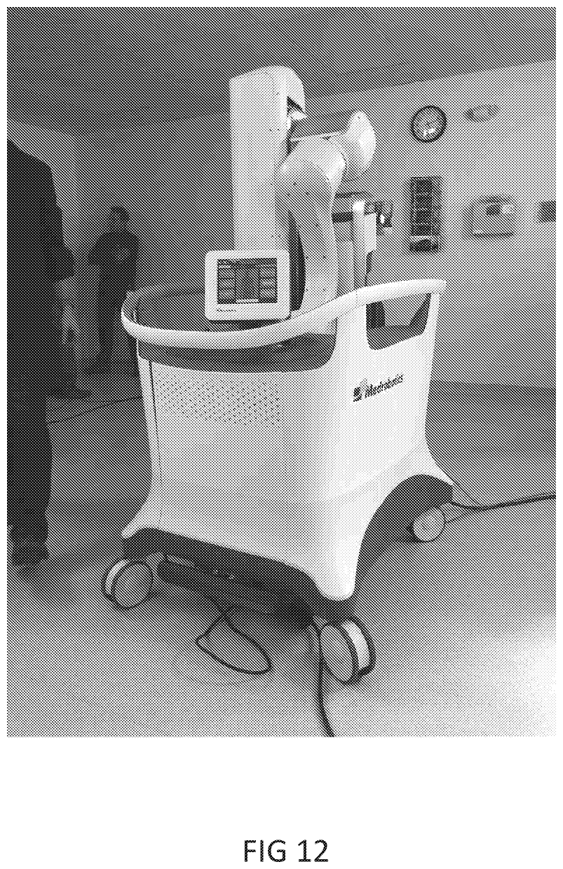

Referring to FIG. 12, a view of the robotic stand 500 in a stowed position is shown, in accordance with embodiments of the present inventive concepts.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for performing a medical procedure on a patient, comprising:
an articulating probe assembly, comprising:
an inner probe comprising multiple articulating inner links;
an outer probe surrounding the inner probe and comprising multiple articulating outer links; and
at least two working channels that exit a distal portion of the probe assembly,
and
at least one tool configured to translate through one of the at least two working channels,
a robotic stand for positioning the articulating probe relative to the patient.

2.-17. (canceled)
